(19) United States
(12) Patent Application Publication
(10) Pub. No.: US 2014/0099384 A1
(43) Pub. Date: Apr. 10, 2014

(54) APPARATUS AND METHODS FOR PRODUCING OZONE

(71) Applicants: Allan Kenneth Frazer Grugeon Hunt, Brough (GB); John George Chewins, Andover (GB)

(72) Inventors: Allan Kenneth Frazer Grugeon Hunt, Brough (GB); John George Chewins, Andover (GB)

(21) Appl. No.: 14/112,517

(22) PCT Filed: May 14, 2012
(86) PCT No.: PCT/GB2012/051067

§ 371 Date: Oct. 17, 2013

(30) Foreign Application Priority Data
May 17, 2011 (GB) 1108263.3

(51) Int. Cl. C01B 13/10

(52) U.S. Cl. CPC C01B 13/10
USPC 424/613; 422/111

(57) ABSTRACT

The invention relates to improvements in apparatus and methods for producing ozone. The apparatus comprises: a differential pressure injector, a means for circulating aqueous fluid through the differential pressure injector and programmable control means. An ozone generator is provided for connection to an oxygen source via an oxygen delivery conduit and a first valve means is located in the oxygen delivery conduit. The ozone generator is fluidly connected to the differential pressure injector via an ozone delivery conduit and second valve means are located in the ozone delivery conduit. Pressure monitoring means are located between the ozone generator and the first valve means for providing a pressure measurement to the control means. The valve means and the fluid circulation means are operable to create a negative pressure in the oxygen and ozone delivery conduits and the pressure measurement is used by the control means to determine the integrity of the oxygen and ozone delivery conduits.

The invention relates to improvements in apparatus and methods for producing ozone.

Ozone is a powerful oxidising gas found in the earth's atmosphere. It can be manufactured by passing a stream of oxygen through a high voltage electrical discharge or through a UV lamp. Ozone can be dissolved into water to create an oxidising solution, which can be used as a biocide to treat a wide range of surfaces. Ozone can be dissolved into water in a number of ways, but most commonly via a bubble diffuser or a differential pressure injector, often referred to as a Venturi injector. Differential pressure injectors create a vacuum as fluid flows through them. The magnitude of this vacuum is dependent on the inlet and outlet pressure of the injector. Differential pressure injectors are used to entrain ozone gas into a fluid and the efficiency of the ozone mass transfer is related to the vacuum. Ensuring a differential pressure injector is working at its optimum mass transfer efficiency is required to produce high concentration aqueous ozone solutions (circa 20 ppm) in a rapid manner.

Ozone generators are supplied with oxygen which is converted into ozone gas. The oxygen can be obtained from a dry air source, or alternatively from a dedicated oxygen supply, such as a canister or cylinder. When using a fixed volume cylinder or canister, the pressure of the oxygen delivered to the ozone generator must be regulated. In small, consumable, fixed volume canisters, the pressure change during oxygen delivery is both continuous and rapid requiring constant regulation of the supply to the ozone generator.

Ozone is a toxic gas, with an Occupational Exposure Limit of 0.1 ppm. Ozone generators can produce gaseous ozone concentrations in the order of tens of thousands of ppm. Ensuring ozone gas produced by an ozone generator is prevented from escaping into the breathable atmosphere is critical.

Differential pressure injectors, such as Venturi injectors, are commonly used in aqueous ozone generating systems as the means for entraining ozone into the fluid. Some examples are as follows.

U.S. Pat. No. 5,151,250 discloses a system that combines an ozone generating means, a Venturi injector, an oxygen source and a fluid with means to pump it through the Venturi injector. The fluid flow through the injector creates a negative pressure downstream of the ozone generator.

U.S. Pat. No. 6,086,833 discloses an ozone based food washing system which uses a pressure gauge for monitoring the supply of oxygen from a fixed volume cylinder to an ozone generator. It also has means to control the pressure downstream of the ozone generator and regulate the supply of ozone gas to the Venturi injector. It does not have means to measure, calculate and optimise the mass transfer efficiency of the injector. The system does not have means to regulate the pressure up-stream (and hence through) the ozone generator.

U.S. Pat. No. 5,431,861 discloses an apparatus for producing “high concentration ozone water solution” of which the maximum concentration referred to is 14 ppm. The system presents an oxygen cylinder source with means to monitor and control the pressure to the ozone generator.

TW-A-200427428 discloses a concept whereby the flow of water generates a negative pressure within a Venturi injector. The negative pressure is detected by a switch and activates an ozone generator, thus delivering ozone to the flowing water.

US-A-2008/0302139 discloses an ozone based laundry system that utilises a negative pressure to ensure that ozone gas does not leak out to atmosphere.

WO-A-2004/103452 discloses an ozone based system for decontaminating surfaces that includes a differential pressure injector, an oxygen source and means to control the pressure from the oxygen source upstream of the ozone generator.

US-A-2005/0061512 discloses a method for heating a fluid using friction. The fluid circulates within a closed loop; however the use of an orifice within the invention would not aid fluid flow control in relation to a differential pressure injector.

CN-A-1557230 discloses a method of using cavitation of a fluid passing through an orifice to induce a high temperature and pressure within the fluid to disinfect said fluid. The orifice does not have a multifunctional user in respect to a differential pressure injector.

One object of this invention is to provide a method and apparatus that allows the rapid generation of a high concentration aqueous ozone solution by optimising the mass transfer efficiency of the differential pressure injector by identifying the magnitude of the negative pressure produced and controlling the amount of oxygen delivered to an ozone generator feeding the differential injector. A further object is to try to ensure no release of ozone gas to the atmosphere, whilst compensating for pressure alterations within the oxygen cylinder supply.

The present invention therefore provides apparatus for producing high concentration aqueous ozone comprising:

a differential pressure injector;

means for circulating aqueous fluid through the differential pressure injector;

programmable control means;

an ozone generator for connection to an oxygen source via an oxygen delivery conduit;

first valve means located in the oxygen delivery conduit;

said ozone generator being fluidly connected to the differential pressure injector via an ozone delivery conduit;

second valve means located in the ozone delivery conduit; and

pressure monitoring means located between the ozone generator and the first valve means for providing a pressure measurement to the control means;

wherein the valve means and the fluid circulation means are operable to create a negative pressure in the oxygen and ozone delivery conduits and the pressure measurement is used by the control means to determine the integrity of the oxygen and ozone delivery conduits.

The control means may be programmed with a minimum negative pressure to be reached when the fluid circulation means are activated with the first valve means closed and the second valve means opened before the ozone generator is activated.

Preferably the control means is programmed to use a maximum negative pressure value measured during a predetermined time period to determine an optimal pressure set-point for the delivery of ozone to the differential pressure injector from the ozone generator to maximise the entrainment of ozone into the fluid.

The first valve means is preferably a proportional solenoid valve, which may used to control the optimal pressure set-point.

The proportional solenoid valve is preferably used to regulate the flow of oxygen into the oxygen delivery line using a control loop based on pressure measurements from the pressure monitoring means.

The apparatus preferably further comprises a fixed volume container as the oxygen source.

Storage means are preferably provided for storing fluid circulated through the differential pressure injector.

The invention additionally provides a method for producing high concentration aqueous ozone comprising the steps of:

creating a negative pressure in a fluid circuit by circulating pressurised fluid through a differential pressure injector in the fluid circuit;

measuring the negative pressure;

using the maximum measured negative pressure to determine an optimal pressure set-point for the delivery of oxygen to the injector via an ozone generator to maximise the entrainment of ozone gas into the fluid;

wherein the measured negative pressure is preferably used to determine the integrity of the delivery lines in the fluid circuit.

Preferably the optimal set-point is below ambient pressure to prevent release of ozone gas to atmosphere.

A proportional solenoid valve is preferably used to control the optimal pressure set-point.

A proportional solenoid valve is preferably used to regulate the flow of oxygen to the ozone generator using a control loop based on the measured pressure.

The present invention thus solves the problem of how to control the flow of oxygen to an ozone generator to produce a high concentration ozone gas and subsequently entrain that gas into a fluid using a differential pressure injector, resulting in a high concentration aqueous ozone solution, the entraining vacuum of the differential pressure injector being dependent on the pressure produced by the pumping means which forces the fluid through the injector; the problem being that the pumping means is variable. The invention further solves the problem described whilst also monitoring the integrity of the ozone gas delivery line to ensure that no ozone gas escapes into the atmosphere.

Implementation of the invention allows for a high concentration aqueous ozone solution to be produced using a differential pressure injector in a fluid line where the pressure upstream or downstream of the injector can vary. The variation in fluid pressure alters the vacuum created by the differential pressure injector which alters the efficiency of the mass transfer of ozone gas into the fluid. The fluid is moved through the differential pressure injector whilst the suction/gas inlet to the injector is fully restricted, producing a defined negative pressure that can be monitored and recorded. A computational algorithm is used to calculate the optimum ozone gas delivery pressure based on the negative pressure generated by the differential pressure injector to maximise ozone mass transfer therefore producing a high concentration aqueous ozone solution as quickly as possible. The ozone gas delivery pressure is controlled by a proportional solenoid valve linked to a pressure transducer. The optimum ozone gas delivery pressure is further controlled to ensure it remains at negative pressure to ambient, ensuring no escape of ozone gas to atmosphere can occur. The control of the ozone gas delivery pressure allows for small fixed volume sources of oxygen to be used as a supply for the ozone generator. The proportional solenoid valve adjusts to keep the oxygen delivery line at the optimum pressure for ozone mass transfer as the pressure in the fixed volume cylinder decreases with time.

One embodiment of the present invention will now be described, by way of example only, with reference to and as shown in the accompanying drawings, in which:

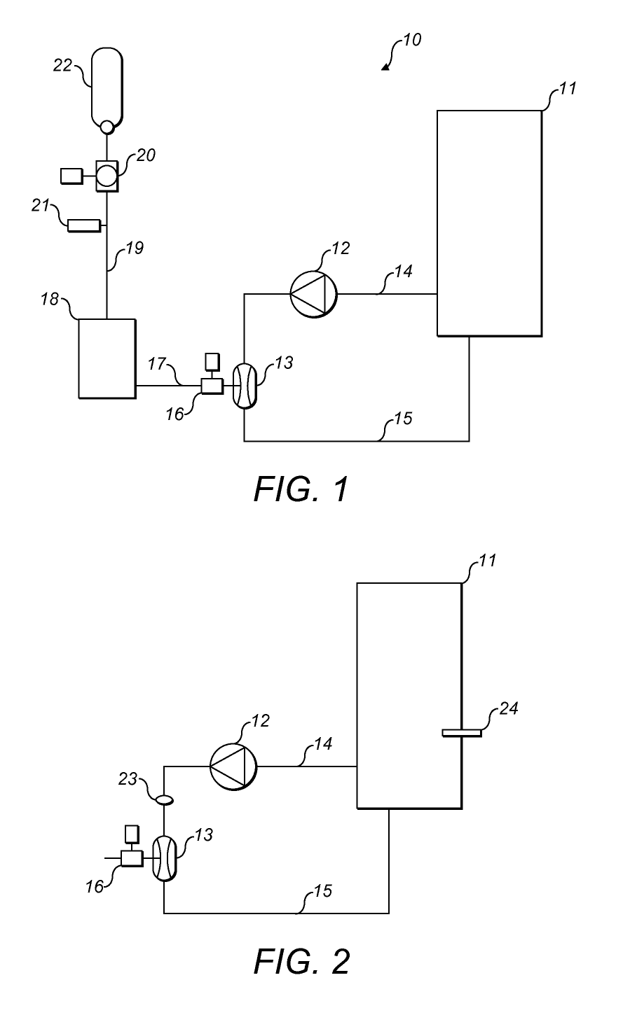

FIG. 1 is a schematic representation of a preferred embodiment of the present invention; and

FIG. 2 is a schematic representation of another embodiment of the present invention incorporating a multi-use orifice.

The present disclosure is based on one particular commercially available differential pressure injector. As such all timings and values described relate to the use of this injector. However the principles of the invention are not limited to the use of this injector and can apply to larger or smaller differential pressure injectors.

FIG. 1 illustrates one arrangement of apparatus 10 that can be used to produce high concentration aqueous ozone fluid. A suitable source of fluid, preferably purified water, is contained within a contact tank 11 and is drawn from the contact tank 11 via conduit 14 by means of a pump 12. The fluid is directed through a differential pressure injector 13 and returns to the contact tank 11 via conduit 15.

A valve 16 is located in a conduit 17 connecting a suction inlet of the injector 13 to an ozone generator 18. The ozone generator 18 has an open flow structure and is linked by means of conduit 19 to a proportional control valve 20. A pressure transducer 21 is located in conduit 19 between the ozone generator 18 and the proportional control valve 20. An oxygen canister 22 is connected to the end of conduit 19 on the other side of the proportional control valve 20.

In use, proportional control valve 20 is closed and when the valve 16 is opened, a negative pressure is produced in conduits 17 and 19.

A countdown timer is activated when valve 16 is opened, the count down being a short period such as 10 seconds. The timer period allows a consistent negative pressure to form in the conduits 17, 19 between the injector 13 and the proportional valve 20. The pressure is monitored by the pressure transducer 21 and a controller records the stabilised value once the count down timer has completed.

In addition, the controller is programmed with a minimum negative pressure value that must be achieved. Failure to reach the minimum value results in the process being discontinued and/or an alarm or warning being given to the operator. The minimum negative pressure value ensures that the conduits 17,19 are free of leaks. If a leak was present, a substantial negative pressure could not be produced and air will be drawn in, preventing escape of ozone gas.

The stabilised negative pressure value is used by the controller in a calculation that relates the maximum or “dead head” negative pressure generated by the flow of fluid through the injector 13 and the corresponding oxygen gas line pressure required to produce the optimal mass transfer/entrainment of ozone gas and hence highest aqueous ozone concentration within a defined time period.

The calculation determines the optimal oxygen gas line pressure required for the stabilised negative pressure generated by the injector 13.

The proportional control valve 20 is then opened to allow oxygen gas to exit the oxygen canister 22. The proportional control valve 20 is controlled by the controller to achieve the calculated optimal gas line pressure using a control loop based on feedback from pressure transducer 21. When the oxygen line pressure has stabilised at the optimal point, the ozone generator 18 is switched on, creating ozone from the oxygen gas flowing through it. The ozone gas is entrained into the fluid by the injector 13.

As oxygen gas exits oxygen canister 22, the pressure within the canister 22 reduces, varying the pressure in the oxygen line. The pressure change is monitored by the pressure transducer 21 and the proportional control valve 20 is adjusted by the controller to retain the optimal oxygen line pressure set-point.

At the point that the controller determines the optimal oxygen line pressure set point using the equation, high and low pressure alarm set points are set based on the calculated figure. For example, if the optimum set point calculated was 800 mbar (abs), the high level pressure alarm would be 810 mbar and the low level pressure alarm 790 mbar. If the pressure within the oxygen line should move outside of these values the system would discontinue operation and/or alarm. These alarm set points allow the system to detect any leaks within the oxygen/ozone gas delivery lines or ozone generator, or failures within the water flow to the differential pressure injector 13.

At the completion of the ozonation phase, the ozone generator 18 is turned off, proportional control valve 20 and valve 16 are closed and pump 12 is turned off.

FIG. 1 presents the preferred embodiment of the invention using a fluid loop on a contact tank 11. This embodiment is preferred as it allows a high concentration solution to be produced. However, the invention is also applicable to an on-line system. In such a system conduits 14,15 are connected to a raw fluid supply and a system to use the ozonated fluid respectively.

To minimise or eliminate pressure fluctuations due to pressure spikes or dips, such as those caused in mains water pressure or due to pump voltage variation, an orifice can be placed up-stream of the differential pressure injector to act as a flow smoothing device. By smoothing the flow to the injector, variations in vacuum pressure are reduced, minimising the changes required to be made by the oxygen line proportional control valve. The orifice can also be used to heat the fluid within the system to a pre-defined temperature. Fluid is moved through the orifice, whilst the gas inlet to the differential pressure injector is closed.

FIG. 2 illustrates the apparatus 10 of FIG. 1 enhanced with a multi-functional orifice 23 and a temperature monitoring apparatus 24. The orifice 23 acts to minimise the effect of any change in the performance of the pump 12, by smoothing the flow of fluid to the differential pressure injector 13. Such pump performance variation could be due to changes in electrical voltage supply or due to mechanical wear. In applications where the aqueous ozone concentration must be carefully controlled, or the surface to which the aqueous ozone is to be applied is temperature sensitive, the temperature monitoring means 24 measures the temperature of the fluid. If it is below the required pre-ozonation temperature, the pump 12 is turned on and fluid is moved through the orifice 23 where it is heated by friction. Valve 16 is closed and the fluid passes through the injector 13 where further, less prolific, temperature change may be imparted to the fluid. The fluid returns to the tank 11 and continues to recirculate through the loop until the temperature monitoring apparatus 24 registers that the pre-determined temperature has been reached.

The use of a flow restricting and smoothing orifice produces a non-electrical heating of the re-circulating fluid.

The multi-functional use of an orifice plate minimise the effect of any pressure fluctuations up-stream of the injector, whilst also acting as a non-electrical, friction based fluid heating means. Heating of the fluid can be beneficial to control the concentration of the aqueous ozone produced. Ozone solubility in water is dependent on temperature; hence controlling the fluid temperature improves the control of the aqueous ozone concentration. Heating the fluid will reduce the maximum possible aqueous ozone concentration. However, some surfaces, such as human skin, are detrimentally affected by biocidal solutions that are too cold, hence the necessity for the fluid to be heated.

1. Apparatus for producing high concentration aqueous ozone comprising:
a differential pressure injector;
means for circulating aqueous fluid through the differential pressure injector;
programmable control means;
an ozone generator for connection to an oxygen source via an oxygen delivery conduit;
first valve means located in the oxygen delivery conduit;
said ozone generator being fluidly connected to the differential pressure injector via an ozone delivery conduit;
second valve means located in the ozone delivery conduit; and
pressure monitoring means located between the ozone generator and the first valve means for providing a pressure measurement to the control means;
wherein the valve means and the fluid circulation means are operable to create a negative pressure in the oxygen and ozone delivery conduits; and
wherein the control means is programmed with a minimum negative pressure which the pressure measurement must reach to ensure that the conduits are free of leaks.

2. Apparatus as claimed in claim 1 in which the control means is the pressure measurement must reach the minimum negative pressure when with a the fluid circulation means are activated with the first valve means closed and the second valve means opened before the ozone generator is activated.

3. Apparatus as claimed in claim 1 in which the control means is programmed to use a maximum negative pressure value measured during a predetermined time period to determine an optimal pressure set-point for the delivery of ozone to the differential pressure injector from the ozone generator to maximise the entrainment of ozone into the fluid.

4. Apparatus as claimed in claim 1 in which the first valve means is a proportional solenoid valve.

5. Apparatus as claimed in claim 4 in which the control means is programmed to adjust the proportional solenoid valve to control the optimal pressure set-point.

6. Apparatus as claimed in claim 4 in which the control means is programmed to adjust the proportional solenoid valve to regulate the flow of oxygen into the oxygen delivery line using a control loop based on pressure measurements from the pressure monitoring means.

7. Apparatus as claimed in claim 1 further comprising a fixed volume container as the oxygen source.

8. Apparatus as claimed in claim 1 further comprising storage means for storing fluid circulated through the differential pressure injector.

9. A method for producing high concentration aqueous ozone comprising the steps of:
creating a negative pressure in a fluid circuit by circulating pressurised fluid through a differential pressure injector in the fluid circuit;
measuring the negative pressure;
using the maximum measured negative pressure to determine an optimal pressure set-point for the delivery of oxygen to the injector via an ozone generator to maximise the entrainment of ozone gas into the fluid;
wherein the measured negative pressure must reach a minimum negative pressure to ensure that delivery lines are free of leaks.

10. A method as claimed in claim 9 wherein the optimal set-point is below ambient pressure to prevent release of ozone gas to atmosphere.

11. A method as claimed in claim 9 wherein a proportional solenoid valve is used to control the optimal pressure set-point.

12. A method as claimed in claim 9 wherein a proportional solenoid valve is used to regulate the flow of oxygen to the ozone generator using a control loop based on the measured pressure
